(19) United States
(12) Patent Application Publication
(10) Pub. No.: US 2007/0038124 A1
(43) Pub. Date: Feb. 15, 2007

(54) Optical probe for arterial tissue analysis

(75) Inventors: Stephen F. Fulghum JR., Marblehead, MA (US); Sudha Thimmaraju, Andover, MA (US); Jonathan Feld, Somerville, MA (US)

Correspondence Address: WEINGARTEN, SCHURGIN, GAGNEBIN & LEBOVICI LLP, TEN POST OFFICE SQUARE, BOSTON, MA, 02109, US

(73) Assignee: Newton Laboratories, Inc.

(21) Appl. No.: 11/445,923

(22) Filed: Jun. 2, 2006

Related U.S. Application Data
(60) Provisional application No. 60/686,600, filed on Jun. 2, 2005.

(51) Int. Cl. A61B 6/00

(52) U.S. Cl. 600/476

(57) ABSTRACT

The present invention relates to systems and methods used in the measurement of arterial tissue. Optical probes in accordance with the invention use optical fibers to deliver and collect light using a sidelooking catheter. Diffused white light and fluorescence scattering is collected and processed to provide for improved artery wall diagnosis.

### CROSS REFERENCE TO RELATED APPLICATIONS

This application claims the priority of U.S. Provisional Application No. 60/686,600 filed Jun. 2, 2005 entitled, OPTICAL PROBE FOR ARTERIAL TISSUE ANAYLSIS, and U.S. Provisional Application No. 60/686,601 filed Jun. 2, 2005 entitled OPTICAL PROBE FOR RAMAN SCATTERING FROM ARTERIAL TISSUE. The entire contents of the above applications are being incorporated herein by reference.

### BACKGROUND OF THE INVENTION

Methods and devices have been developed for the diagnosis of arterial tissue. These methods have included spectroscopic techniques such as tissue autofluorescence to measure characteristics of the tissue. Among those characteristics to be measured are included the presence of vulnerable plaques that contribute to the susceptability of individuals to heart disease or stroke. More specifically, these vulnerable plaques can result in the formation of blood clots that can cause heart attacks or stroke.

Fiber optic catheters have been developed to deliver and collect light within the vascular system for the purpose of diagnosing vascular disease. For measurements within the coronary arteries this requires the use of small diameter probes to access the arterial wall in which vulnerable plaques may form. Vulnerable plaques typically have a fibrous cap overlying a lipid tissue formation. There remain difficulties however in the rapid and reliable measurement of such plaques within the vascular system. A continuing need exists, however, for further improvements in systems and methods for reliable arterial diagnosis.

### SUMMARY OF THE INVENTION

Angioscopy is of value in visualizing atherosclerotic lesions in a vascular field flushed with saline or other clear fluid. Angioscopy has indicated that yellow color intensity of the plaque is strongly related with the prevalence of thrombosis on the plaque. The yellow color intensity can thus be used as a marker of plaque vulnerability. Calibrated white light reflectance measurements based on broadband sources, grating spectrometers and detection of the spectra can provide a more precise color analysis and are reliable in identifying vulnerable plaques in real time.

In addition to the morphological information obtained, a preferred embodiment of the present invention employs a complementary system and method for probing and detecting the biochemical information of plaques. Flexible optical fibers can be used to deliver light to the tissue and rapidly collect spectroscopic signals, so that tissue can be evaluated without removal. Fluorescence spectroscopy can be used to provide diagnosis and analysis of atherosclerosis in combination with reflectance measurements.

Interior artery wall tissue fluorescence exhibits differences in its fluorescence spectra depending on whether it is normal, atherosclerotic, atheromatous or calcified atherosclerotic plaque. In the present invention UV and visible laser excitation wavelengths can be used with a classification program with high sensitivity and specificity. The programs derive their capability from the differing fluorescence spectra of collagen and elastin fibers, lipoproteins and oxidized lipopigments such as ceroid, and attenuation of fluorescence spectra by carotenoids that are often present in the atheroma of atherosclerotic tissue.

Tissue differentiation of vulnerable plaques can be performed using three dominant fluorophores: elastin, collagen, and 3h-oxLDL, which had differentiable fluorescence spectra that can be used as a basis set enabling deconvolution of measured tissue spectra, to determine the relative concentration of these constituents. The relative concentrations then enabled differentiation between normal artery, atheroma, and the lipid pool of vulnerable plaque. The data were also interpreted in terms of the thickness of the fibrous cap over the lipid pool. Improvements in diagnostic accuracy employ the methods of Wu, et al. (See U.S. Pat. No. 5,452,723, the entire contents being incorporated herein by reference.) to obtain the intrinsic fluorescence (i.e. the native fluorescence of the tissue embedded fluorophores, unencumbered by spectral distortions produced by absorbers interacting with the diffusely scattering light). Intrinsic fluorescence spectroscopy requires simultaneous fluorescence and diffuse (white light) reflectance spectroscopy measurements.

The present invention employs simultaneous fluorescence and reflectance spectroscopy along with intrinsic fluorescence measurements on the tissue.

As diffuse reflectance measurements alone enable a quantifiable method for color judgment, and color is an indicator of the twin characteristics of a thin fibrous cap over a significant lipid pool (yellow) as a marker of vulnerable plaque. The present invention can simultaneously gather the reflectance data to implement intrinsic fluorescence methods to diagnose vulnerable plaque.

A clinically effective optical fiber probe to measure artery fluorescence in blood vessels is preferably capable of providing a well-defined geometry within the artery wall, minimizing any blood in the optical path, and providing circumferential/azimuthal differentiation. A side-looking probe is particularly advantageous in the small diameter, confined geometry of arteries.

The present invention relates to a side-looking optical probe, such as a catheter, to detect fluorescence and diffuse white light scattering from artery walls. A preferred embodiment, the probe utilizes an axially symmetric structure with a lumen centered on the longitudinal axis so that the probe can optionally be inserted over a guidewire which has been previously placed into the artery. In a preferred embodiment, four optical fibers, equally spaced around the central lumen, transmit excitation light to the distal tip. A reflective optical element such as a sapphire axicon at the distal tip of the probe directs this excitation light sideways to the tissue. Within each quadrant, a plurality of optical fibers receives fluorescence and scattered white light from the tissue and transmits it to the proximal end of the probe for analysis. Both the excitation fibers and collection fibers are placed at a single radial distance from the central axis. The axicon is polished in an elliptical, toroidal shape which focuses the ends of the fibers onto the tissue within a plane passing through the central axis of the probe.

The toroidal shape of the axicon causes the light from the fibers to diverge azimuthally from the four excitation fibers, leading to a ring-shaped illumination profile on the tissue. The receiver fibers for a given quadrant are placed on either side of the excitation fiber for that quadrant. Their effective collection area thus overlaps the illumination area for that quadrant. The received spectra from the four quadrants are preferably independent and cover the complete circumference of the artery. By continuously monitoring the four spectra as the probe is withdrawn a complete map of the arterial wall is obtained without rotation of the probe.

### BRIEF DESCRIPTION OF THE DRAWINGS

FIG. 1 illustrates a system for arterial tissue diagnosis in accordance with a preferred embodiment of the invention.

FIGS. 2A-2G are illustrations of an optical probe in accordance with a preferred embodiment of the invention.

FIGS. 3A-3D illustrate an optical coupling system for a probe in accordance with a preferred embodiment of the invention.

FIG. 4 is a detailed perspective view of a bifurcation assembly of an optical probe in accordance with a preferred embodiment of the invention.

FIGS. 5A-5C graphically illustrate reflectance spectral characteristics of vulnerable plaques with yellowing of fibrous caps under 100 microns.

FIGS. 6A-6D illustrate fluorescence characteristics of vulnerable plaque.

FIG. 7 illustrates an example of a decision plot for diagnosis of a vulnerable plaque using a fluorescence and diffuse reflectance process.

FIGS. 8A-8F illustrate another preferred embodiment of an optical coupling system.

FIGS. 9A and 9B illustrate a reflective optical element in accordance with a preferred embodiment of the invention.

### DETAILED DESCRIPTION OF THE INVENTION

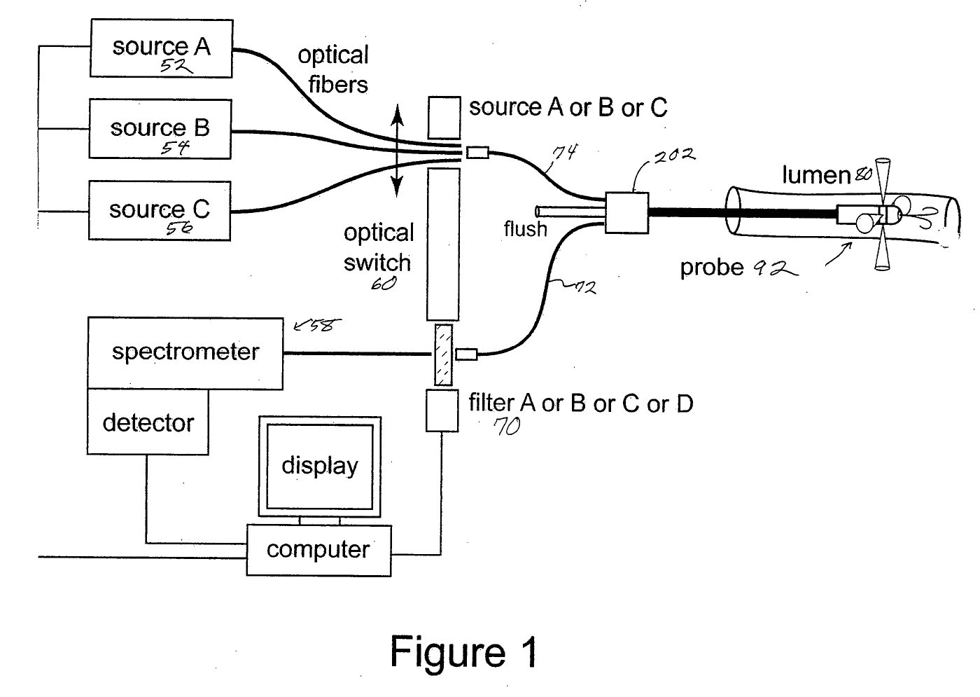

FIG. 1 shows a schematic of the diagnostic system. Three excitation sources can be used. An ultraviolet (UV) excitation source (A), operating at 337 nm from a nitrogen laser, for example, a visible excitation range in the range of 400 nm to 700 nm from a diode laser (B), for example, and a broadband white light excitation from a source (C) such as a xenon arc lamp or quartz tungsten halogen bulb for example. The UV and visible lasers are chosen to excite particular fluorophores in the tissue. The white light is used to determine the scattering characteristics of the tissue including its apparent color. An optical switch 60 can be used to select which of the light sources is coupled to the excitation delivery fibers 74 in the probe 92 at a given moment and simultaneously moves the appropriate optical filter 70 into the receiver fiber 72 path before the spectrometer 58.

In the case of the UV or visible fluorescence diagnostics these filters are used to block excitation light reflected from the tissue from entering the spectrometer and saturating the detector. In the case of the white light diagnostic these filters are used to modify the “white” light spectrum so that the system response, including the detectors for the dispersed spectrum, is relatively flat, leading to a preferred signal to noise ratio across the visible spectrum. A fourth filter, comprising a clear window, is positioned in the spectrometer path when no source is coupled into the probe so that background light can be measured and used for processing such as subtraction from the other measured spectra. The optical probe is a separate component that can be attached to the system after disinfection or sterilization. It includes a bifurcated input/output section 202, a two-way delivery section of sufficient length to reach the tissue of interest in lumen 80 and a distal tip containing the axicon element to direct the excitation sideways and to couple the tissue response back into the receiver fibers.

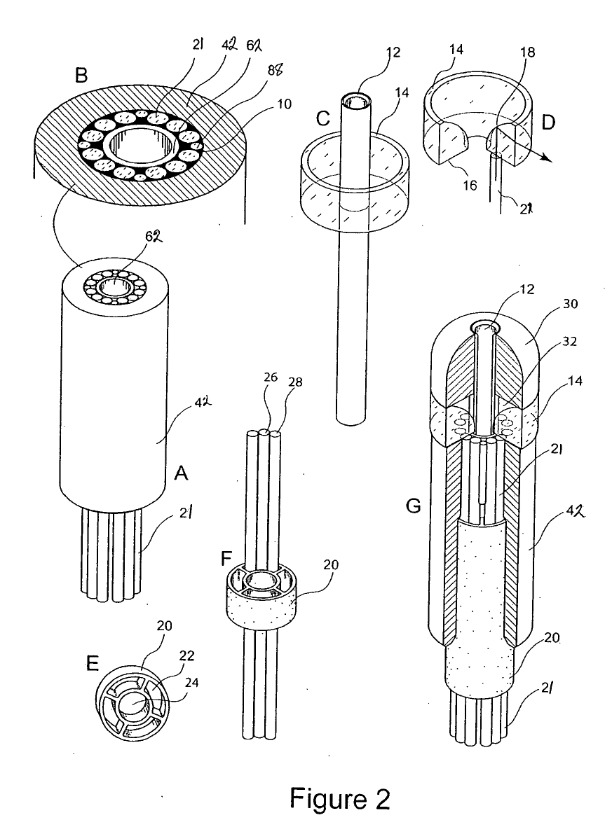

The structure of the distal tip is shown in FIGS. 2A-2G. The fiber assembly is shown in FIG. 2A. The delivery fibers, 2, are held between a heavy-wall tube of stainless steel, 4, on the outside and a thin-walled tube of stainless steel, 6, on the inside. To divide the fibers into quadrants, small fibers or wires, 8, can be used as spacers and a black epoxy, 10, is used to hold the fibers in place so that they can be optically polished to a common plane perpendicular to the fiber axes as shown in the fiber assembly tip detail, FIG. 2B.

FIG. 2C shows a thin-walled stainless steel core tube, 12, which is used to provide alignment for the axicon, 14, to the fiber assembly. This core tube slips both into the central tube, 6, of the fiber assembly and into the central bore of the sapphire axicon. Besides alignment, this core tube provides shear strength for the entire assembly and a passageway for the guide wire in the assembly probe

FIG. 2D shows the axicon, 14, with a quadrant cut away to illustrate its elliptical shape. A preferred material for the axicon, 14, is sapphire because of its hardness and because of its high index of refraction which reduces the angular spread of the excitation light exiting the optical fibers. A clear optical epoxy such as EPO-TEX 310 is used to bond the core tube, 12, and axicon, 14, to the fiber assembly and to provide an index match between the fused silica fibers, 2, and the back surface, 16, of the sapphire axicon. The arrow in FIG. 2D shows one optical path that light can take from a delivery fiber, 2, to the tissue. This path is totally internally reflected from the front surface of the axicon at position 18 if there is an air space left behind the axicon. The preferred embodiment will use an axicon coated with a thin metallic layer, such as aluminum, on surface 18 so that the space behind that surface can be filled with an epoxy sealant.

FIG. 2E shows a section of a long, multi-lumen, extruded tube, 20, used to carry the optical fibers to and from the distal tip in the outer lumens, 22, while keeping them separated and sealed from the core lumen, 24. The core lumen, 24, carries the guidewire and saline flush for the probe. This type of custom extruded tube is supplied by Zeus, Inc. among others. FIG. 2F shows the position of the excitation fiber, 26, for one quadrant flanked on either side by receiver fibers, 28.

FIG. 2G shows the assembled probe tip in which the multi-lumen tube, 20, has been slipped over the optical fibers, 20, and bonded to the core tube, 12, and the outer tube, 4, to complete the distal seal of the lumens, 22, carrying the fibers from the central lumen, 24, exposed to saline flush and body fluids. The sapphire axicon, 14, is protected by a rounded tip, 30, bonded to the core tube, 12, which may also be formed to partially fill or fill the volume, 32, which is otherwise sealed with epoxy. The rounded tip, 30, which also serves to protect the arterial walls from abrasion during insertion of the probe, may also be formed from an epoxy bead to combine the sealant and protective functions.

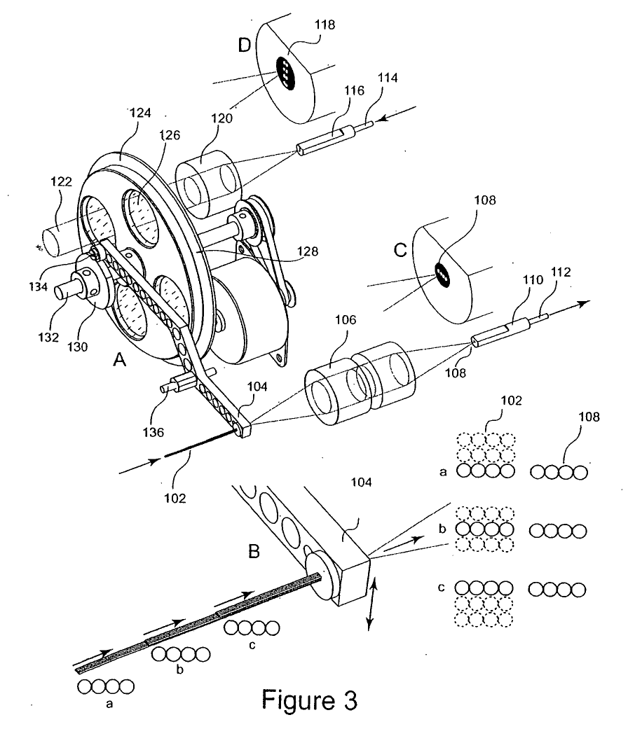

FIGS. 3A-3D show a preferred embodiment of an optical switch used to couple in the three light sources, narrowband UV excitation, narrowband visible excitation and broadband white light into the four fibers used to carry the excitation light to the distal tip of the probe. Each of the three light sources are coupled to the switch with a band of four optical fibers forming a rectangular array of three rows of four columns, 102, which are coupled to switches at the end of movable arm 104. The fibers have sufficient length between the sources and the coupling to the optical switch that they are free to move a short distance up and down without significant bending stresses being developed. FIG. 3B shows the end of movable arm, 104, in greater detail. The light exits the polished ends of the fiber array, 102, and is collected by achromatic lens set 106 which images this array onto a single row of four fibers, 108, held in a specialized ferrule 110 leading to one arm of the bifurcated probe bundle 112. FIG. 3C shows in detail how the row of four probe fibers are held in the ferrule face, parallel to the rows of the source fibers. The ferrule, 110, has a polished front face and a registration flat so that different probes can be attached to the optical switch with the excitation fibers held in the same position at the focal plane of lens set, 106. The ends of the light source array, 102, are maintained in the opposite focal plane of the lens set, 106. Only one source fiber row at a time can be imaged onto the probe fiber row so the selection of the source illumination in the probe depends on the position of the movable arm 104. The nominal fiber diameters are only 0.2 mm, so that only a small vertical motion of one fiber diameter is necessary to switch light sources. This small motion can be effected quickly and without significant stress to the fibers. Overlap of the light from the other sources due to possible imaging errors in the lenses is not an issue because the light sources are only switched on after the motion has been completed and they are not typically switched on simultaneously.

Light from the tissue is returned to the optical switch through optical fibers carried in the other arm of the bifurcated probe bundle, 114. These fibers are arranged in a vertical array, 118, so that they can be imaged onto the entrance slit of an imaging spectrograph which reimages the fiber array onto a 2D, pixellated detector with dispersion for recording the spectrum of the returned light. The fibers from each of the four quadrants are physically separated from each other in this array, as shown in FIG. 3D, so that the spectra from each of the four quadrants can be read out from the 2D detector and recorded separately. Lens set 120 collimates the light from the fiber array, 118, into a beam 122, which passes through an aperture in rotating wheel 124 containing a filter, 126, appropriate to that source. For the fluorescence excitation sources this filter is an excitation light blocking filter which passes the weaker fluorescence. For the white light source this filter can be a clear window or a spectrum modification filter for smoothing the response of the 2D detector. The filtered and collimated beam, 122, is focused directly onto the entrance slit of the spectrometer which is wide enough to accommodate the width of the image of the fiber array, 118.

A small slot is cut into the rim of the rotating filter wheel, 124, for purposes of triggering the light sources at the correct time when a filter is in the correct position. This wheel can either rotate continuously or be controlled by a stepper motor, depending upon the length of time that a filter needs to be in position. In a preferred embodiment the filter wheel rotates several times per second so that a complete spectral sequence of fluorescence, white light, fluorescence, dark can be recorded several times per second as the probe is drawn though a body lumen. The appropriate position for the movable arm, 104, and thus the choice of the light source to the probe, is controlled by a cam, 128, attached to the axle of the rotary wheel, 130. A rolling cam follower, 132, is attached to the movable arm, 104, which is pivoted on shaft 134. The position of the movable arm pivot is nominally set for a 2:1 ratio of cam follower motion to fiber bundle motion. A nominal 0.2 mm fiber diameter thus has a 0.4 mm land on the. cam to properly position the correct source when the appropriate filter is in position. Note that this arrangement does not require special timing or fixed rotation rates for the wheel. All sequencing can be related to a signal derived from the timing notch, 124.

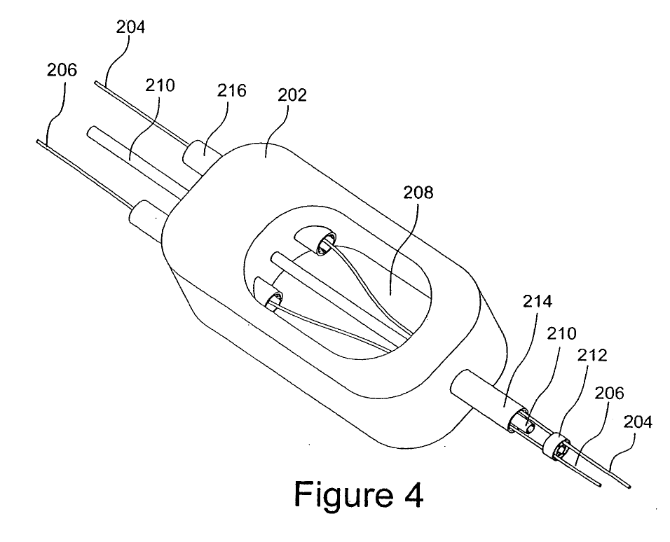

FIG. 4 shows the coupled 202, at the bifurcation of the probe which serves to combine the excitation fibers, 204, (shown for clarity as a single fiber) and reception fibers, 206, (also shown as a single fiber) within a cavity, 208, which are filled with potting compound. The fixture, 202, also serves to provide access for a guide tube, 210, which can be bonded into the central lumen of the multi-lumen probe tube, 212, shown as a short section. This guide tube, 210, provides access for the guidewire and saline flush to the probe. The outer wall of the multi-lumen tube is bonded to the inner wall of the connector tube, 214, for pull strength, sealing against body fluids and sealing against fluids during sterilization. Similarly, connector tubes, 216, are used to seal to the outer wall of the jackets surrounding the excitation fibers and reception fibers in their respective bifurcated bundles. The potting compound placed into cavity 208 seals these fiber jackets against fluids as well.

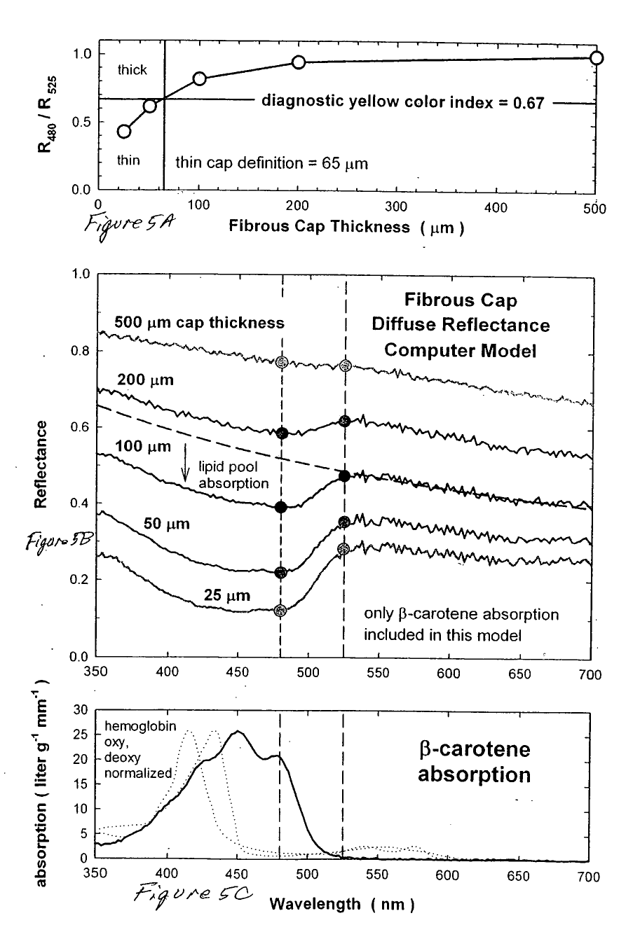

For visible light, a fibrous cap can turn incident light around within about 100 μm. If the fibrous cap is very thick its apparent color is white because the collagen in the cap does not absorb visible wavelengths. If the fibrous cap is much thinner than 100 μm then visible light can reach the lipid pool where the blue photons will be absorbed by beta-carotene. (See FIGS. 5b and 5C). A portion of the green and red photons (making yellow) will eventually scatter back out giving the vulnerable plaque its distinctive yellow color.

If a UV excitation photon is incident on a thick fibrous cap it will also be largely turned around before it reaches the lipid pool. The only visible fluorescence will be blue from the collagen within the cap itself. If the cap is thin there will be less blue fluorescence as more UV photons make it to the lipid pool. The primary component of the pool, oxidized LDL, may then fluoresce and emit photons at blue and green wavelengths. The blue photons from the oxidized LDL will be absorbed by the beta-carotene in the pool so that only the green photons can escape (shifting its fluorescence peak to the red). The increasing green fluorescence, relative to the blue fluorescence, is thus and indicator of thinner caps.

Even though the incident light is white (all colors of equal intensity) the return signal is somewhat blue. This is because the scattering particles are larger relative to blue wavelengths so that blue photons are scattered more strongly than red photons. Stronger scattering means that blue photons return to the surface of the tissue more quickly and are more likely to be within the tissue area from which the probe can collect light. At 200 μm in this example very few photons make it through the lipid pool so there is only a slight reduction in the blue wavelengths reflected back. At 100 μm the reflected spectrum has lost its blue tinge and is essentially white. At 50 μm a significant fraction of the photons make it to the lipid pool and the tissue becomes visibly yellow. At 25 μm the yellow color is even stronger. An important point to note is that the observed absorption in tissue scattering is not linear. Photons do not take a fixed path. Many paths are very long so that even a small absorption can be significant. The “noise” at red wavelengths in all of the spectra is actually weak noise in the absorption data shown in FIG. 5C. It is particularly noticeable for λ>525 nm where it is no longer small relative to the average absorption.

The sharp absorption edge of the beta-carotene absorption around 500 nm, enhanced by the absorption saturation effect of the long, random path, tissue scattering, provides a possible diagnostic for the yellow color associated with vulnerable plaques. By taking the ratio of the reflectivity at 480 nm to the reflectivity at 525 nm, a single number, R₄₈₀/R₅₂₅, can be obtained to quantify “yellow”. This ratio is small for thin caps and large for thick caps so it retains the sense of an indicator of cap thickness. Ratios are particularly useful in optical probe diagnostics because the absolute reflectance signals will vary with distance from the tissue while the relative shape of the spectrum will tend to remain constant. The ratios in this method are plotted in FIG. 5A along with a horizontal line at 0.67 representing the index associated with a cap thickness of 65 μm. For a probe with arterial tissue, the optimal wavelengths for the ration can be different and the numerical results might shift but be accurate as an indicator of “yellow”.

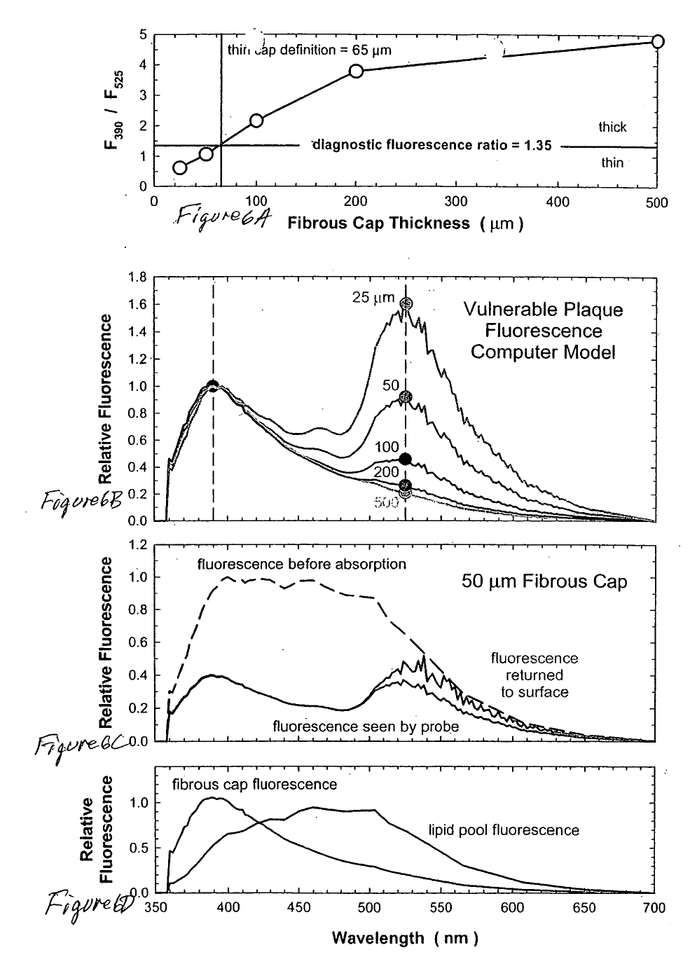

The fluorescence method can use three wavelengths: (1) the excitation wavelength of 337 nm, (2) the peak fibrous cap fluorescence wavelength at 390 nm and (3) the peak lipid pool fluorescence wavelength at 490 nm. An example of this method is shown in FIGS. 6A-6D. The relative fluorescence efficiencies of the fibrous cap and lipid pool have been chosen to illustrate the method. These input parameters can be determined from ex vivo measurements on carotid endarterectomy samples.

In the fluorescence method excitation photons at 337 nm are scattered by the arterial tissue and convert to fluorescence photons. Absorption in each of the two layers due to hemoglobin and beta-carotene slowly reduces the “intensity” and this the level of fluorescence that is can produce. If a conversion happens to occur in the top layer representing the fibrous cap then the wavelength considered for the scattering characteristics changes to 390 nm and a new path length begins to be summed. If the conversion occurs in the bottom layer the scattering characteristics are defined by 490 nm.

It is important to note that even though the lipid pool fluorescence spectrum is also generally “blue/green”, the spectrum which escapes the lipid pool and thus through the non-absorbing fibrous cap is distinctly yellow due to absorption by beta-carotene. only a few of the paths resulting in lipid pool fluorescence occur at the interface and exit immediately. The double fluorescence peaks in the method results provide another opportunity for a ratio measurement which can be a diagnostic of the fibrous cap thickness. In this case it is a ratio between fluorescence at 390 nm and fluorescence at 525 nm. The inverse can be used but it is convenient to maintain the same sense that a small parameter represents a thin cap so F₃₉₀/F₅₂₅ is chosen. The value for this method at the 65 μm definition of this is 1.35 for example.

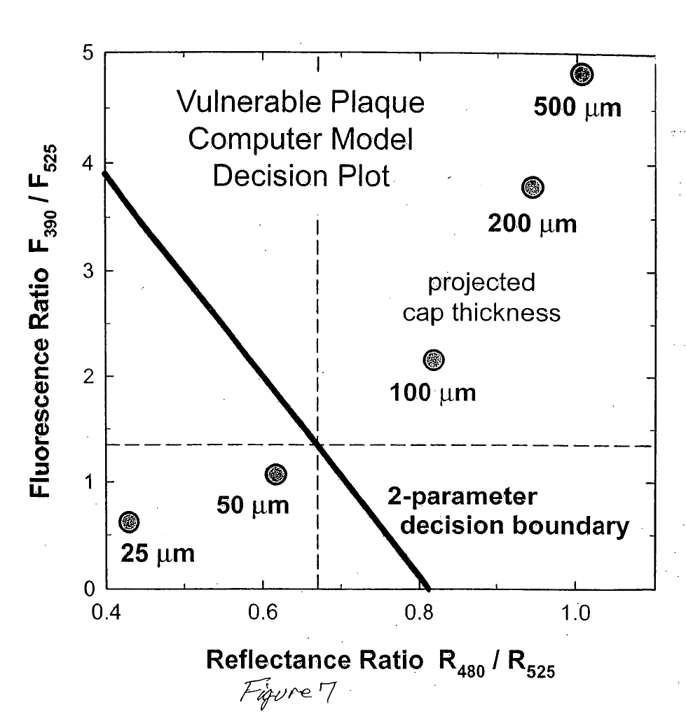

Using two different physical processes to measure one physical quantity gives an advantage in terms of the specificity and sensitivity of a diagnostic. A convenient way to plot the results in the a 2-map with each of the parameters as an axis. Such a plot if present in FIG. 7 along with the data points that this particular example of the method indicates for the different thicknesses of the fibrous cap. In tissue data there is noise on the measurements and interference from other physical properties which will vary from tissue site to tissue site and patient to patient. In general, however, there is a line that can be drawn across the 2-D map similar to the one shown in FIG. 7 which will best separate measurements which have been divided by pathology into “thick caps” or “thin caps”.

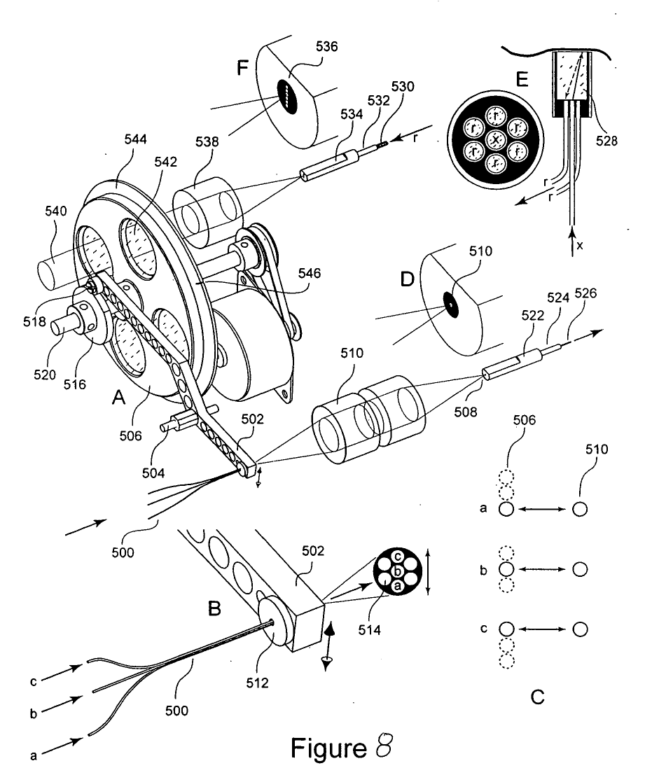

FIGS. 8A-8F show a preferred embodiment of the optical switch as used to couple three light sources, (a) narrowband UV excitation, (b) broadband white light, and (c) narrowband visible excitation into a central, forward-looking probe delivery fiber. For the forward-looking probes the side-directing axicon is not necessary and is replaced with a simple window to set the probe offset distance from the tissue. A ring of receiver fibers, typically six in number, carry the fluorescence and reflected white light back to the optical switch and spectrometer. The optical switch method of coupling light sources into the delivery fiber avoids the losses of dichroic beamsplitters which are often used for combining optical sources onto a common axis. The optical switch allows wavelengths which are very close together to be combined which is particularly difficult with dichroic beamsplitter methods. Laser sources can often be combined by illuminating a high NA optical fiber with a narrow-angle beam. However, it is inefficient to couple thermal white light sources together with lasers using that method. The optical switch method efficiently couples any set of fiberoptic sources. The optical switch method also eliminates the necessity of carefully positioning sources relative to each other since the light is carried to the switch through flexible optical fibers.

Only one source fiber row at a time can be imaged onto the probe fiber row so the selection of the source illumination in the probe depends on the position of the movable arm 104. The nominal fiber diameters are only 0.2 mm, so that only a small vertical motion of one fiber diameter is necessary to switch light sources. This small motion can be effected quickly and without significant stress to the fibers. Overlap of the light from the other sources due to possible imaging errors in the lenses is not an issue because the light sources are only switched on after the motion has been completed and they are not switched on simultaneously.

FIG. 8A shows the three fiberoptic source fibers, 500, attached to the movable arm of the optical switch, 502, which pivots about the shaft, 504 to produce an oscillating vertical motion at the end of the movable arm determined by the rotary position of the filter wheel 506. Each source fiber is imaged, in turn, onto a single delivery fiber, 508, by the lens set 510. The delivery fiber carries the light from each source fiber, in turn, to the distal tip of the forward-looking probe. The optical switching of the source fiber light into the delivery fiber is thus accomplished mechanically and is locked in phase with the filter wheel.

FIG. 8B shows an enlarged view of the end of the movable arm or fiber coupler, 502, and the ferrule, 512, holding the source fibers in their proper positions. The three fibers are aligned into a single column by close-packing them with dummy fibers, 514, and bonding them in place. After bonding the source fiber ends are polished so that their light can be launched into free space towards the imaging lens set, 510. The ferrule is rotated, before being locked into place with respect to the movable arm, so that the three source fibers are aligned along the axis of motion of the movable arm.

FIG. 8C shows how the three source fibers are sequentially switched in position so that they can be imaged onto the probe delivery fiber. The neutral position of the movable arm occurs twice in one rotation of the filter wheel and holds source fiber “b” in the imaging (light delivery) position. Continued rotation of the filter wheel brings a high or raised section of the cam, 516, in FIG. 8A under the cam follower, 518, which moves the end of the movable arm down bringing source fiber “c” into the light delivery position. Further rotation brings source fiber “b” back into position. Generally, a spectrum can be taken without a source light to measure the background light entering the spectrometer. This second position can be used for that purpose. Alternatively, any source resulting in a weak signal can be placed in this position to achieve two exposures during one rotation sequence. Further rotation brings a low, or depressed segment of the cam, under the cam follower resulting in an upward motion of the end of the movable arm bringing source fiber “a” into the delivery position. The cycle then repeats. Note that the angular position of the cam, 516, relative to the filter wheel shaft, 520, relates the filter wheel position to the source fiber position. This cam can be part of the filter wheel or directly attached to it to fix that phase relationship.

The movable arm shown in FIG. 8A shows a 2:1 mechanical reduction relating the high and low positions on the cam to the spacing of the fibers at the tip of the movable arm. A nominal 0.25 mm fiber spacing (assuming a typical cladding around a standard 0.2 mm diameter core) thus requires a 0.5 mm high and low positions on the cam to properly position the correct source fibers relative to the wheel position.

Only one source fiber at a time is imaged onto the probe delivery fiber. The nominal 0.25 mm motion of the movable arm can be effected quickly and without significant stress on the source delivery fibers. Overlap of the light from the other sources due to aberrations in the imaging lenses is not an issue because the light sources are only switched on after the motion has been completed and they are not switched on simultaneously.

The polished proximal end of the delivery fiber, 508, in FIG. 8A is carried in a ferrule 522 designed to be lockable into a single, fixed and repeatable position. This ferrule is part of a removable forward-looking probe assembly. A jacket, 524, protects the single delivery fiber 526 from physical damage.

FIG. 8E shows a simplified view of a forward-looking probe with a central delivery fiber marked as “x” surrounded by six receiver or collection fibers marked “r”. A window allows the light to expand from the delivery fiber to cover an area of tissue larger than the delivery fiber itself. This window also provides for an overlap between the illuminated area of the tissue and the tissue area “seen” by the receiver fibers.

Fluorescence and reflected light collected by the receiver fibers is carried back to the optical switch by a fiber bundle, 530, for filtering. This bundle is protected by a jacket, 532, and realigned into a vertical array within the receiver ferrule, 534, which has a flat to maintain its rotational alignment at the switch. The linear array of receiver fibers, 516, is shown in the enlarged FIG. 8F.

In FIG. 8A the collimating lens set, 538, is held at one focal length from the linear array of fibers, 536, collimating the received light into a single beam, 540. This beam is passed through the appropriate filter, 542, carried in the filter wheel, 506. The four filters carried by the wheel differ depending on the light source they are matched with by the optical switch. For the fluorescence excitation sources this filter would be an excitation light blocking filter which would only pass the weaker fluorescence and prevent saturation of the spectrometer. For the white light source this filter could be an anti-reflection coated clear window.

A thin web on the rim of the filter wheel, 544, is slotted at one point, 546, for the purpose of optically generating a timing pulse for triggering the light sources at the correct time when a filter is in the correct position. The filter wheel can either rotate continuously or be controlled by a stepper motor, depending upon the length of time that a filter needs to be in position. In a preferred embodiment the filter wheel rotates several times per second so that a complete spectral sequence of white light reflectance, fluorescence 1, dark background and fluorescence 2 can be recorded several times per second.

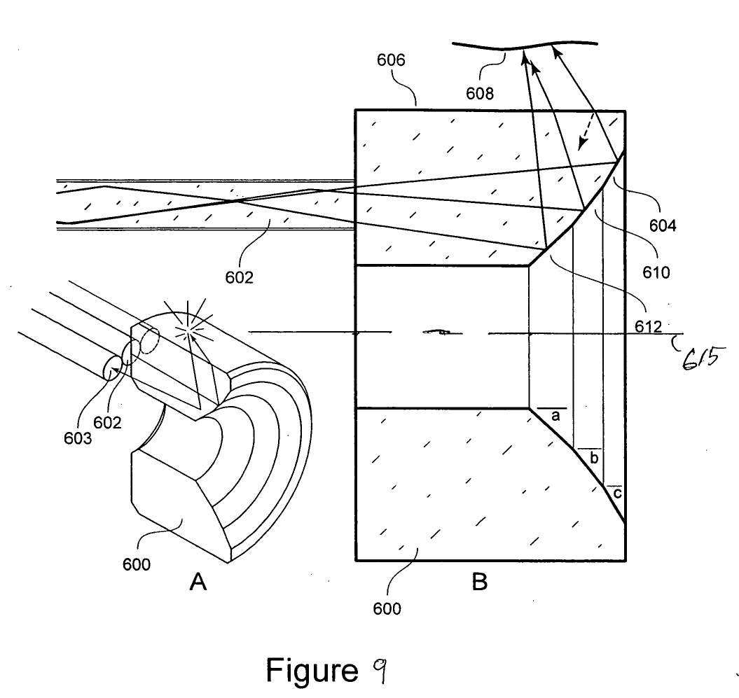

FIGS. 9A and 9B illustrate a preferred embodiment of a reflective optical element such as an axicon used for the side-looking probe. In this embodiment plurality of flat reflective surfaces are used to approximate an elliptical surface. In this particular embodiment three surfaces or facets are used. This simplifies the manufacture of the component without significantly degrading the optical performance. FIG. 9A shows an isometric view of the axicon, 600, cut in half to better illustrate the angles used. FIG. 9B shows an orthographic projection of the same axicon, 600, along with the radial position of one of the delivery fibers, 602, and adjacent collection fibers, 603. Solid lines in FIG. 9B show typical ray traces used in the design of the axicon. The line intersecting the facet face, 604, reflects off of that face and intersects the outer rim of the axicon, 606, well off of normal incidence. The dashed line at that ray intersection shows the Fresnel reflection from surface 606. By keeping the input rays generally angled backwards towards the delivery fiber Fresnel reflections are prevented from following the incoming path backwards and entering the receiver fibers. This reduces noise in the received light signals from the tissue, 608. Facet faces 610 and 612 are also chosen to prevent back reflections into the receiver fibers. Nominal angles, “a”, “b” and “c” are 44 degrees, 52 degrees and 60 degrees relative to longitudinal axis 615 of the probe distal end. Keeping the overall tilt of these angles to within ±2 degrees and a relative tilt between adjacent angles to ±1 degree is sufficient to prevent most of the back reflections. Note that the facets 604, 610 and 612 must be coated with a reflective material, such as aluminum, since the angles will not support total internal reflection. Such a coating also allows the tip of the fiberoptic probe to be filled with a material such as epoxy to simplify manufacture and avoid pockets of contamination.

The claims should not be read as limited to the described order or elements unless stated to that effect. Therefore all embodiments that come within the scope and spirit of the following claims and equivalents thereto are claimed as the invention.

What is claimed is:

1. An optical probe for arterial tissue comprising:
a fiber optic probe having at least one delivery optical fiber and a plurality of collection optical fibers;
a reflective optical element to deliver and collect light in a radial direction at a distal end of the probe;
a first light source to deliver fluorescence excitation light;
a second light source to deliver reflectance light;
a filter element; and
a detector system that detects the fluorescence and reflectance light.

2. The optical probe of claim 1 further comprising a third light source.

3. The optical probe of claim 1 further comprising an optical fiber switch.

4. The optical probe of claim 1 further comprising a filter switch.

5. The optical probe of claim 1 further comprising a data processor that processed fluorescence and reflectance data to provide a diagnostic indicator of fibrous cap thickness.

6. The optical probe of claim 1 wherein the reflective optical element comprises a first reflective surface at a first angle and a second reflective surface at a second angle.

7. The optical probe of claim 1 wherein the reflective optical element comprises a curved surface.

8. The optical probe of claim 1 wherein the reflective optical element comprises a plurality of flat surfaces at different angles.

9. The optical probe of claim 3 wherein the switch comprises a moveable element that couples light from a first light source fiber or a second light source fiber to the delivery fiber.

10. A method of using an optical probe for arterial tissue comprising:
providing a fiber optic probe having at least one delivery optical fiber and a plurality of collection optical fibers;
providing a reflective optical element to deliver and collect light in a radial direction at a distal end of the probe;
coupling light from a first light source to deliver fluorescence excitation light with at least one delivery fiber;
coupling light from a second light source to deliver reflectance light;
providing a filter element; and
detecting fluorescence and reflectance light with the collection optical fibers.

11. The method of claim 10 further comprising providing a third light source.

12. The method of claim 10 further comprising providing an optical fiber switch.

13. The method of claim 10 further comprising providing a filter switch.

14. The method of claim 10 further comprising processing spectral data with a data processor that processed fluorescence and reflectance data to provide a diagnostic indicator of fibrous cap thickness.

15. The method of claim 10 further comprising actuating the optical fiber switch to couple light from a first light source fiber into the delivery optical fiber.

16. The method of claim 15 further comprising actuating the optical fiber switch by moving a fiber coupler to align a second light source fiber into the delivery optical fiber.

17. The method of claim 16 further comprising actuating the optical fiber coupler to align a third light source optical fiber with the delivery fiber.

18. The method of claim 18 further comprising actuating three sources in sequence in less than one second while actuating the optical fiber switch.

19. The method of claim 10 further comprising reflecting light with a plurality of surfaces on the reflective optical element, the surfaces being positioned at different angles relative to a longitudinal axis of the distal end of the probe.

20. The method of claim 19 further comprising reflecting light off at least three surfaces in a radial direction.
